(19) United States
(12) Patent Application Publication
(10) Pub. No.: US 2025/0188494 A1
(43) Pub. Date: Jun. 12, 2025

(54) A METHOD OF CELL ELECTROPORATION

(71) Applicant: MAXCYTE, INC., Rockville, MD (US)

(72) Inventor: Peter David GEE, Uji City, Kyoto (JP)

(73) Assignee: MAXCYTE, INC., Rockville, MD (US)

(21) Appl. No.: 18/838,851

(22) PCT Filed: Feb. 16, 2023
(86) PCT No.: PCT/US2023/013245

§ 371 Date: Aug. 15, 2024

Related U.S. Application Data
(60) Provisional application No. 63/311,006, filed on Feb. 16, 2022.

(51) Int. Cl. C12N 15/87; C12N 5/074; C12N 9/22; C12N 13/00; C12N 15/11

(52) U.S. Cl. CPC C12N 15/87; C12N 5/0696; C12N 9/22; C12N 13/00; C12N 15/111; C12N 2310/20; C12N 2523/00; C12N 2800/10

(57) ABSTRACT

Method of electroporating with cell culture aimed to improve cell viability wherein the method comprises electroporating cells of interest with an anti-apoptosis protein, treating the cells with DNase post-electroporating, and shifting the temperature of the cells from 37° C. to 32° C. while resting post electroporation.

### PRIORITY

This application claims priority to U.S. Provisional Application No. 63/311,006 filed on Feb. 16, 2022, which is incorporated herein by reference in its entirety.

### FIELD

The present disclosure relates generally to methods to enhance cell viability and transgene expression from a large DNA plasmid into cells, such as iPS cells, in a novel combination approach of different enhancer methods for cell viability and transgene expression from an exogenous large DNA plasmid.

### BACKGROUND

Electroporation is a known method of introducing compositions into cells. The terminology of electroporation, electro-transfection, and electroloading have been used interchangeably in the literature with emphasis on the general meaning of this technology. The electroporation may be, for example, flow electroporation or static electroporation. Methods and devices for electroporation are also described in, for example published PCT Application Nos. WO 03/018751 and WO 2004/031353; U.S. patent application Ser. Nos. 10/781,440, 10/080,272, and 10/675,592; and U.S. Pat. Nos. 5,720,921, 6,074,605, 6,773,669, 6,090,617, 6,485,961, 6,617,154, 5,612,207, all of which are incorporated by reference.

Electroporation can damage cells leading to a decrease in cell viability post-electroporation. The prior art does not describe an electroporation method or system that improves cell viability and expression when trying to transfect large plasmids, such as DNA. The present invention presents a method of electroporating cells that increases cell viability.

The present disclosure seeks to overcome the foregoing deficiencies by a method that could be applied for other cell types and applications to improve the efficiency of cell engineering whether it is related to protein production or cell therapy. Accordingly, there is described a novel way to improve cell viability and expression of very difficult-to-transfect large DNA plasmids for accomplishing cellular engineering.

The disclosed methods seek to overcome one or more problems of the prior art by enhancing cell viability and transgene expression from a large DNA plasmid into cells, such as iPS cells. Thus, there is described a balanced electroporation protocol with appropriate DNA concentration which allows for cells to survive by adding the enhancers. The enhancers have been shown to have no effect if the DNA is too toxic and the electroporation energy is too high.

### SUMMARY

In one embodiment there is disclosed a method of electroporation with cell culturing that includes the following steps: a) co-electroporating cells with an anti-apoptosis protein, such as an mRNA form of the gene BCL-XL; b) adding DNase to the electroporated cells that are at a temperature T1, such as above 37° C.; and c) decreasing the temperature of cells after electroporation to a temperature T2, such as 32° C. or below.

In one embodiment, there is described a method to co-electroporate BCL-XL mRNA with DNA plasmids in iPSCs. As used herein iPS cells mean “induced pluripotent stem cells”. These are a type of pluripotent stem cell derived from adult somatic cells that have been genetically reprogrammed to an embryonic stem (ES) cell-like state through the forced expression of genes and factors important for maintaining the defining properties of ES cells.

In one embodiment, there is described modifications to electroporation protocols to achieve the beneficial properties described herein. Such modifications include, but are not limited to, adding BCL-XL mRNA to improve cell viability and enhance transgene expression. One or more of the described modifications results in increased cell stability. This requires a balance between higher efficiency and less cells versus lower efficiency and higher cell number.

In one embodiment, there is described post-electroporation modifications, which includes but is not limited to; adding DNase after electroporation enhances cell viability; and cold shock, such as for 48 hours seems to enhance transgene expression.

In one embodiment, the method further comprising adding a gene editing tool being delivered as a DNA plasmid with the DNase. Non-limiting examples of the gene editing tool that can be used herein include CRISPR-Cas9 based PRIME editing or base editor.

In one embodiment, the DNase is added in an amount of 1 unit of DNase is added per 20 ul of volume.

Temperature shifting to a lower temperature reduces the degradation of delivered cargo and reduces cell proliferation which ensures higher expression of the transgene from the vector delivered into the cell.

In one embodiment, after BCL-XL mRNA is co-electroplated with DNA plasmids in iPSCs, DNase can be added to electroporated cells, and subsequently recovered to allow for membrane recovery. Finally, the disclosed method includes a thermal treatment step, which typically comprises lowering the temperature, such as to 32° C. for a desired period of time, such as 24-48 hours, before increasing the temperature to about 37° C.

An electroporated cell comprising at least one added material, made by the disclosed method is also described. The disclosed electroporated cell comprising an added material that enhances at least one property selected from cell viability and transgene expression.

### BRIEF DESCRIPTION OF THE DRAWINGS

The accompanying drawings, which comprise a part of this specification, illustrate several embodiments and, together with the description, serve to explain the principles disclosed herein. In the drawings:

FIG. 1 shows the bright field images/results from treating iPSC cells with DNase two days after electroporation.

FIG. 2 shows both GFP fluorescence microscope images and bright field images of iPS cells electroporated with three different conditions.

FIG. 3 shows both GFP fluorescence microscope images and bright field images of iPS cells electroporated with four different conditions.

### DETAILED DESCRIPTION

As used herein iPS cells means “induced pluripotent stem cells”. These are a type of pluripotent stem cell derived from adult somatic cells that have been genetically reprogrammed to an embryonic stem (ES) cell-like state through the forced expression of genes and factors important for maintaining the defining properties of ES cells.

As used herein, “passage number” is the number of times a cell culture has been subcultured, i.e. harvested and reseeded into multiple ‘daughter’ cell culture flasks.

As used herein, “DNase” refers to Deoxyribonuclease, which is a major nuclease present in the blood and other body fluids. It is responsible for the digestion of extracellular nucleoproteins, which may be crucial for the prevention of autoimmune reactions.

Unless specifically defined otherwise herein, all technical, scientific, and other terms used herein have the same meaning as commonly understood by one of ordinary skill in the art of ratings-based methods and web-based reputation systems and related sciences. Additional terms may be defined, as required, in the disclosure that follows.

The chosen cells or cell line of interest are expanded and/or stimulated for a designated length of time pending on the cell type. On the day of electroporation, cells, suspension or adherent, are harvested and a cell sampling is taken for cell counts and viability.

In one embodiment, the cell culturing includes dissociating the target cells.

In one embodiment, single-cell dissociation for cell culturing works and is better for counting cells more accurately and determining the cell viability.

In one embodiment, cells are washed with a buffer or basal medium to remove any residual components in the medium. The chosen cell number are condensed by centrifugation or any cell condensing apparatus pending on the scope of the experiment to the processing assembly.

In one embodiment, cell culture can be completed by using techniques known in the art specific to the cell type of interest. The cells can be passaged via clump passaging with EDTA or a single cell passage using Accutase. In one embodiment, the concentration of cells after culturing ranges from 1×10⁷-5×10⁷ cells/mL.

In one embodiment, cells are dissociated with ethylene diamine tetra-acetic acid (EDTA), as used herein, EDTA refers to a cell detachment solution used for clump passaging.

In one embodiment, cells are dissociated in accutase. As used herein “accutase” refers to a cell detachment solution containing proteolytic and collagenolytic enzymes.

In one embodiment, cells are treated with DNase after they are electroporated. DNase allows for cell membrane recovery after electroporation. Post electroporation, excess DNA outside of the cell membrane prevents the membrane from resealing. Removing the excess DNA helps the cell close pores that were opened during electroporation. Any commercially available DNAse can be used according to the embodiments described herein, such as Pulmozyme (Genentech).

In one embodiment, an anti-apoptosis gene can be added to increase cell viability. Non-limiting examples of such genes include BCL2 or BLC-XL. The methods disclosed herein, may be used with a wide variety of cell populations. In some embodiments, the cells may be from blood, interstitial fluid, and any tissues, such as bone marrow, peripheral blood, or cord blood, or any other normal or tissues affected by a disease.

In one embodiment, the cells may be from whole peripheral blood or whole cord blood. In some embodiments, the cells may be from whole peripheral blood mononuclear cells (PBMCs). In some embodiments, the cells may be from whole cord blood mononuclear cells (CBMCs). In some embodiments, the cells may be from a fraction of peripheral blood mononuclear cells (PBMCs). In some embodiments, the cells may be from a fraction of cord blood mononuclear cells (CBMCs). In some embodiments, the cells may be from a specific cellular component of the blood.

Non-limiting examples of PBMCs include alpha beta TCR+ T cells, gamma delta TCR+ T cells, NK cells, invariant NKT cells, B cells, dendritic cells, monocytes, macrophages, neutrophils, granulocytes, hematopoietic progenitor cells, mesenchymal progenitor cells, and stromal cells. These cells may be mature or immature cells. These cells may also be lineage committed and noncommitted cells.

In one embodiment, the cell type can be chosen from but is not limited to T cells, HSCs, NK cells, DCs, B cells, PBMCs, HEK293s, CHOs, IPSC cells, and/or K652s.

In one embodiment, the claimed method can be used with iPSC cells. To do so with a single cell passage, one could passage the cells at a low cell number and expand for 7-8 days (e.g. 15-20,000 cells in a 6-well plate; 1.5-2.0×10³ cells/cm²). This leads to highly compact colonies where the cells in the center of the colony are denser than the cells in the outer part of the colony by day 7-8, which may lead to lower homogenous electroporation efficiency.

In one embodiment, one could passage the cells at a higher density, for example, if trying to obtain 80% confluency by passaging 3 days before electroporation one can seed 100,000 cells in a 6-well plate which would be 1×10⁴ cells/cm². If passaging is done the day before electroporation, passage the cells at 1×10⁶ cells in a 6-well plate which would be 1×10⁵ cells/cm². One skilled in the art would understand that these are approximate cell seeding densities and need to be adjusted based on the growth properties of the iPSCs. Some iPSCs will grow faster depending on the coating and medium used. For example, iMatrix-511 combined with StemFit AK03N media will generally reach 80% confluency with the above conditions. With other combinations of coating and media, the kinetics of cell proliferation may be different.

In one embodiment, the cells can be passaged three days before electroporation using a dissociation reagent such as 0.5 mM EDTA, at 1:10 ratio. For instance, from a single well in a 6-well plate, seed 10% of the well into a new well of a 6-well plate. Generally, this will lead to approximately 80% confluency on the day of electroporation. Cell viability should be >90% for iPSC lines. In general cells with a passage number less than 30 are preferred and the doubling rate should be <24 hours. Cells that have been just thawed after cryopreservation should not be electroporated. Cells should be passaged 2-3 times after cryopreservation and the cell doubling rate should be confirmed prior to electroporation.

In one embodiment, iPSC cells can be prepared for electroporation by first washing the cells with PBS, aspirating the PBS, repeating the wash step if cells are floating in suspension, adding 0.5 mL of accutase to the well of a 6-well plate or an appropriate amount of accutase to cover the plate if using a larger culture vessel, rocking the plate to ensure the surface is covered with accutase, placing the plate into a 37° C. incubator for 5-15 minutes but depending on the coating agent used, the cells may take a long time to round and be ready for dissociation from the plate.

For example, in one embodiment, there is used a product to allow for cell adhesion, such as a protein. In one embodiment, when using a protein based coating (vitronectin coating), cells tend to come off within 5 minutes. However, with i-Matrix-511, it sometimes takes more than 10 minutes for the cells to start to dissociate. Next, pipette the cells up and down in the accutase solution in order to get single-cell dissociation. It is desired that one does not use a scraper to mix the cells at this point as it will damage the cells.

In some embodiments, the method may comprise next adding the iPSCs with accutase to a centrifuge tube containing iPSC media. For example, if a 6-well plate is used, 0.5 mL accutase pipetted cells would be added to a 1.5 mL centrifuge tube containing 0.9 mL of iPSC media. If multiple wells are being combined, then a larger centrifuge tube can be used.

In one embodiment, the method next comprises centrifuging the cells at 250× gravity (g)—for 5 minutes with a slow break. Caution should be taken not to stress the cells during centrifugation as high centrifugation speeds could sensitize the cells to electroporation damage. For example, centrifuging at >300 gs may lead to damaging the iPSCs. After centrifugation, aspirate the supernatant and pipet any residual media out of the tube without disturbing the cell pellet. Then resuspend cells in 5-10 mL of electroporation buffer using standard washing techniques. Next, count the cells and measure viability to ensure viability is not below 80%.

After counting the cells, the method may next comprise centrifuging the cells at 250×g for 5-10 minutes with a slow break. While the cells are centrifuging, a processing assembly packaging (PA) was sprayed down with 70% isopropyl alcohol (IPA), take the PA packaging into a biosafety cabinet and open the packages.

In some embodiments, it is possible to prepare 1.5 mL microcentrifuge tubes corresponding to the DNA concentration which will be evaluated and pipette the corresponding amount of DNA into the 1.5 mL microcentrifuge tubes. For large DNA it is best to use between 50-300 ug/mL total DNA concentration. Once the microcentrifuge tubes are prepared, electroporation may occur. This comprises removing the cells from the centrifuge and aspirate the supernatant. Resuspend cells in the appropriate amount of electroporation buffer to obtain the desired cell concentration. Take the cell pellet volume into account. For example, if 1 mL of the total volume is needed, the cell volume may be 300 ul, so add only 700 ul of buffer to bring the total to 1 mL. The cell pellet volume can be estimated by comparing an empty 50 ml tube and filling it with a known volume. Or, less volume can be intentionally added to the cells so that the total volume is less than 1 mL. Then the appropriate amount of volume can be added to bring the total to 1 mL. Estimation at this stage is acceptable, however, be aware that understanding the cell density will help with ensuring the reproducibility of experiments.

After the buffer is added, aliquot the appropriate volume of cells to the 1.5 mL centrifuge tubes which contain the desired amount of DNA and 50 ug/ml of BCL-XL mRNA. Mix the cells and DNA at least 5 times. Avoid introducing bubbles. Then transfer the cells and loading agent mixture in a processing assembly, place the processing assembly onto an electroporation system, and electroporate the cells.

After electroporation, transfer the cells from the processing assembly into a v-bottom 96-well plate containing DNase. Rinse the well of the processing assembly out with an equal volume of MaxCyte buffer and transfer it to the same well of the 96-well plate. Use 1 unit of DNase for every 20 ul of volume. Mix the solution in a humidified incubator at 37° C. with 5% CO₂ for 30-40 minutes to recover. Transfer cells from the processing assembly to 37° C. prewarmed culture media in a culture vessel.

The processing assembly can be rinsed with media to retrieve any residual cells and added to the culture vessel. The cell density after electroporation will depend on cell survival. For instance, if choosing an electroporation energy that prioritizes efficiency over viability, there will be fewer cells surviving so more cells should be seeded into the culture vessel. If a lower electroporation energy with higher cell survival is selected, then a lower seeding density should be chosen. Finally, culture the cells at 32° C. for 1-2 days before shifting the temperature back to 37° C. Reducing the temperature will slow down cell proliferation and enhance gene expression.

With reference to the figures,

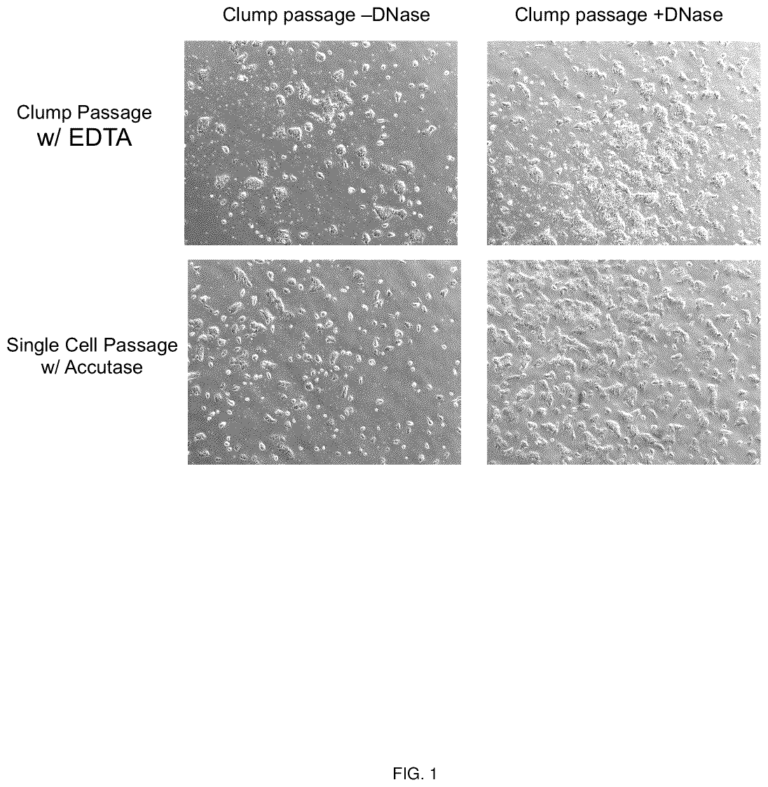

FIG. 1 shows the bright field images/results from treating iPSC cells with DNase two days after electroporation. The figure shows results from both a single-cell passage using accutase and a clump passage using EDTA. The accutase treatment was used to improve cell dissociation which made it easier to determine cell viability and cell count. In both the clump and single-cell passage, treating the cells with DNase post-electroporation improved cell viability. The figure demonstrates this by showing a higher concentration of cells in the images for cells treated with DNase

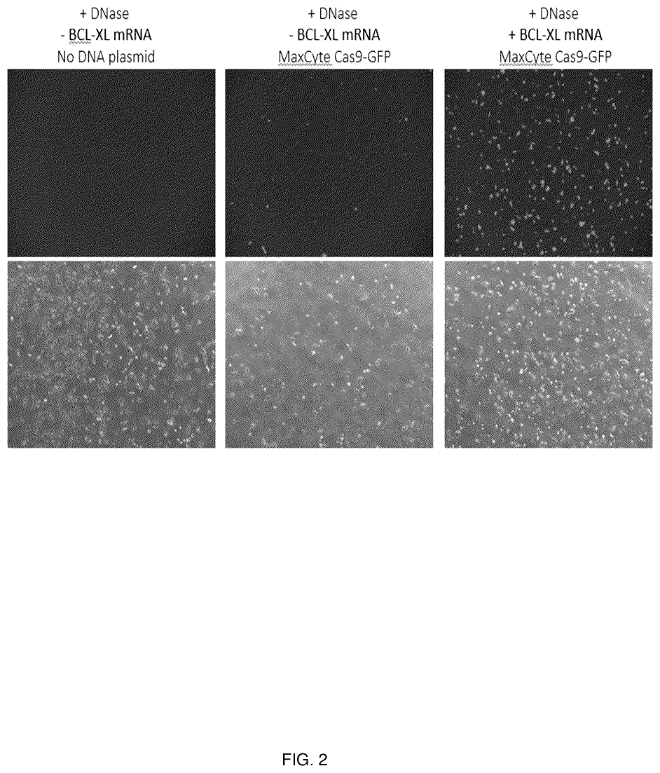

FIG. 2 shows both GFP fluorescence microscope images and bright field images of iPS cells electroporated with three different conditions. Column 1 shows iPS cells that were electroporated without DNA plasmid and BCL-XL mRNA. iPS cells were treated with DNase post-electroporation. No GFP expression is observed and cells looked healthy 24 hours after electroporation. Column 2 shows iPS cells that were electroporated with a large DNA plasmid, expressing a Cas9 and GFP genes, and without BCL-XL mRNA. The iPS Cells were treated with DNase post-electroporation. Weak GFP expression was observed and cell viability was poor 24 hours after electroporation. Column 3 shows iPS cells that were electroporated with a large DNA plasmid, expressing a Cas9 and GFP genes, and BCL-XL mRNA. The iPS Cells were treated with DNase post-electroporation. Higher expression of GFP and cell survival were observed 24 hours after electroporation

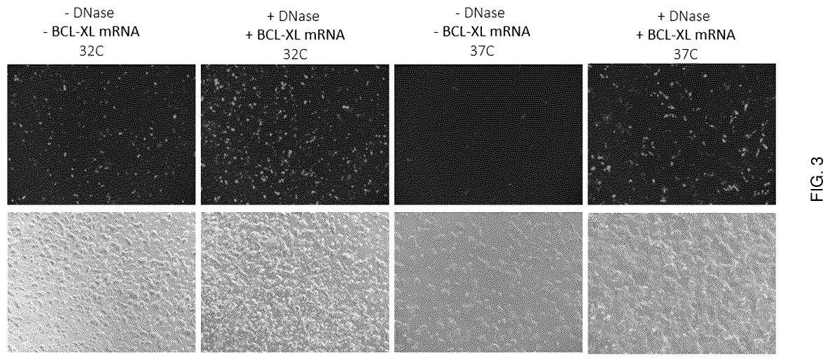

FIG. 3 shows both GFP fluorescence microscope images and bright field images of iPS cells electroporated with four different conditions. Column one shows iPS cells that were electroporated with a large DNA plasmid, expressing a Cas9 and GFP genes, and no BCL-XL mRNA. The iPS cells were not treated with DNase post-electroporation and cultured at 32 C for 48 hours. Column 2 shows iPS cells that were electroporated with a large DNA plasmid, expressing a Cas9 and GFP genes, and BCL-XL mRNA. The iPS cells were treated with DNase post-electroporation and cultured at 32 C for 48 hours. Column 3 shows iPS cells that were electroporated with a large DNA plasmid, expressing a Cas9 and GFP genes, and no BCL-XL mRNA. The iPS cells were not treated with DNase post-electroporation and cultured at 37 C for 48 hours. Column 4 shows iPS cells that were electroporated with a large DNA plasmid, expressing a Cas9 and GFP genes, and BCL-XL mRNA. The iPS cells were treated with DNase post-electroporation and cultured at 37 C for 48 hours. The highest expression of GFP was observed in column 2 when BCL-XL mRNA was co-transfected with DNA, iPS cells were treated with DNase post electroporation, and cultured at 32 C for 48 hours. This is demonstrated by the higher concentration of GFP positive cells in column 2 compared to the other columns.

In some embodiments, the spatial and temporal control of electroporation efficiency may be altered or adjusted within a population of cells. It is contemplated that various specific certain parameters can be applied to the transfecting method that would have an effect on one cell type but not on the other, such as affecting T cells rather than affecting B cells within a sample of cells from a subject.

In some embodiments, the methods and compositions disclosed herein may be effective in many immunotherapies, including, but not limited to, for the treatment of cancer and autoimmune diseases. The methods and compositions disclosed herein may also be used for treatment in several other diseases, including but not limited to, chronic diseases and infections, a viral infection, a bacterial infection, or a parasitic infection, Graft-versus-Host disease, lymphoproliferative disorders, and hyperproliferative diseases. It is contemplated that these methods and compositions may be useful for additional indications not discussed herein.

In some embodiments, the modulation is direct or indirect. In some embodiments, the alteration is direct or indirect. In some embodiments, the therapeutic effectiveness or therapeutic index may encompass an immune response, an immune activation, or an immune suppression.

In one aspect of the present disclosure, methods of generating modified cells for in vitro or ex vivo cellular vaccine therapy are provided. The methods include the steps of isolating cells, introducing a composition into the cells, and administering the cells to a subject. In some embodiments, the composition comprises at least one mRNA encoding at least one antigen, either alone or in combination thereof, wherein the modified cells may induce or are capable of inducing an immune response against the antigen. In some embodiments, the modified cells may induce or are capable of inducing an immune response against other antigens expressed by the target cell in the subject through a mechanism called epitope spreading.

In some embodiments, the gene editing agent includes CRISPR CAS-9, RNA, plasmid, mega-TALS, gene-writing, DNase I, Benzonase, Exonuclease I, Exonuclease III, Mung Bean Nuclease, Nuclease BAL 31, RNase I, 51 Nuclease, Lambda Exonuclease, RecJ, T7 exonuclease, zinc finger nuclease, meganuclease, transcription activator-like effector nuclease, or site-specific nuclease.

The term “cellular vaccines” as used herein refers to cells modified to express antigens. In particular, cellular vaccines refer to cells modified to induce immune responses against an antigen and activate immune cells against the target antigen expressing cells. The cellular vaccines if delivered to a subject and generate inflammatory milieu and elicit immune responses against malignancy, and against abnormally proliferating autoimmune cells, cells infected with viruses, bacteria, fungus, or any disease causing biological agents, thereby providing them the ability to specifically suppress and/or inactivate or kill the diseased/infected or disease causing cells. Nonlimiting examples of antigens may include proteins, polypeptides, carbohydrate antigens, lipoproteins, or peptide antigens, or peptidomimetic.

In general, molecules may include proteins, nucleotide sequences, carbohydrates, lipoproteins, or fragments thereof. Any of these molecules may be used as an antigen or used to produce an antigen, for example, in the case of the nucleotide sequence. These molecules may be natural (i.e., biological) or synthetic. In some embodiments, an antigen may be a protein, a polypeptide, a peptide multimer, a peptide avimer, a carbohydrate antigen, or a lipid protein, or a combination thereof.

The term “transduction” is used to describe a virus-mediated transfer of nucleic acids into cells. In contrast to transfection of cells with foreign DNA or RNA, no transfection reagent is needed here. The viral vector, itself, also called a virion, is able to infect cells and transport the DNA directly into the nucleus, independent of further action. After the release of DNA into the nucleus, the protein of interest is produced using the cell's machinery.

The features and advantages of the methods disclosed herein are illustrated by the following examples, which are not to be construed as limiting the scope of the present disclosure in any way.

### Example

To demonstrate the success of the claimed method, iPSC cells were prepared by growing them to a density of 80% confluency after 32 passages. Once the cells reached a final density of (x), they were spun down in a centrifuge at 250×g for 5 minutes with a slow break. While the cells were in the centrifuge, an electroporation system (here a ExPERT STx OC-25×3 processing assembly) was sprayed down in a biosafety cabinet (BSC) with 70% isopropyl alcohol. A 1.5 mL microcentrifuge tubes corresponding to 100 ug/mL DNA concentration and with 50 ug/mL of BCL-XL mRNA was prepared. Then the electroporation system was turned on and the Opt 4 protocol was selected. After the cells finished spinning down in the centrifuge, they were moved into the BSC and sprayed with IPA before aspirating the supernatant. The pellet was then resuspended in MaxCyte buffer to obtain a cell density of 1×10⁸ cells/mL. From the mixture, 20 ul of volume was added to the 1.5 mL centrifuge tube along with 100 ug/mL of DNA, and 50 ug/mL BCL-XL mRNA. The cells were then gently mixed and transferred to the processing assembly, which was then placed onto the MaxCyte (x) and electroporated. 24.

After electroporation, the cells were transferred from the processing assembly into a 96-well plate. The well of the processing assembly was washed out with an equal volume of buffer and transferred to the same well of the 96-well plate. Then the 40 ul was split into two wells (20 ul into each well, with 1×10⁶ cells per well). Then 2 units of DNase (pulmozyme; dornase alpha) was added. The solution was then added to a humidified incubator at 37° C. with 5% CO₂ for 30 minutes to recover. Post recovery, the cells were transferred from the 96-well plate to a 37° C. prewarmed culture media in a 6-well culture vessel and cultured at 32° C. for 24 hours. The bright field and fluorescence microscopy was used to analyze the results.

FIG. 3 shows that the cells that were (1) electroporated with BCL-XL mRNA, (2) treated with DNase post-electroporation, and (3) experienced a temperature shift from 37° C. to 32° C. post-electroporation, exhibited the highest expression of GFP when compared to cells electroporated without BCL-XL mRNA that also underwent a temperature shift.

It is to be understood that both the descriptions disclosed herein are merely illustrative and intended to be non-limiting.

The specification and examples disclosed herein are intended to be considered as exemplary only, with a true scope and spirit of the invention being indicated in the claims. Other embodiments of the compositions, devices and methods described herein will be apparent to those skilled in the art from consideration of the disclosure and practice of the various example embodiments disclosed herein.

Other than in the examples, or where otherwise indicated, all numbers expressing quantities of ingredients, reaction conditions, analytical measurements, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

As used herein the terms “the,” “a,” or “an” mean “at least one,” and should not be limited to “only one” unless explicitly indicated to the contrary. Thus, for example, “a hybrid peptide” should be construed to mean “at least one hybrid peptide.”

All publications, patents, and patent applications mentioned in this specification are herein incorporated by reference in their entirety into the specification to the same extent as if each individual publication, patent or patent application was specifically and individually indicated to be incorporated herein by reference.

What is claimed is:

1. A method of electroporation with cell culturing, said method comprising:
(a) co-electroporating cells with an anti-apoptosis protein to form electroporated cells;
(b) adding DNase to the electroporated cells that are at a temperature T1; and
(c) decreasing the temperature of cells after electroporation to a temperature T2.

2. The method in claim 1, wherein the cell culturing includes dissociating cells.

3. The method in claim 1, wherein the anti-apoptosis protein is an mRNA form of the gene BCL-XL.

4. The method in claim 3, wherein the BCL-XL is in plasmid form.

5. The method in claim 1, wherein the anti-apoptosis protein is an mRNA form of the gene BCL2.

6. The method in claim 5, wherein the BCL2 is in plasmid form.

7. The method in claim 1, wherein the cell type is selected from the group consisting of iPSC, PBMCs, B cells, HEK293 cells, and CHO cells.

8. The method in claim 1, further comprising adding a gene editing tool being delivered as a DNA plasmid with the DNase.

9. The method of claim 8, wherein the gene editing tool is a CRISPR-Cas9 based PRIME editing or base editor.

10. The method of claim 1, wherein 1 unit of DNase is added per 20 ul of volume.

11. The method of claim 1, wherein temperature T1 is above 37° C.

12. The method of claim 1, wherein temperature T2 is 32° C. or below.

13. The method of claim 12, wherein the temperature is maintained at T2 for a period ranging from 30-40 minutes.

14. An electroporated cell comprising at least one added material, said added material enhancing at least one property selected from cell viability and transgene expression, wherein said electroporated cell is made by the method of claim 1.
